(19) United States
(12) Patent Application Publication
(10) Pub. No.: US 2015/0071946 A1
(43) Pub. Date: Mar. 12, 2015

(54) TUMOR-SPECIFIC RETROTRANSPOSON INSERTIONS

(71) Applicant: The Johns Hopkins University, Baltimore, MD (US)

(72) Inventors: Szilvia Solyom, Baltimore, MD (US); Haig H. Kazazian, JR., Baltimore, MD (US)

(21) Appl. No.: 14/478,419

(22) Filed: Sep. 5, 2014

Related U.S. Application Data
(60) Provisional application No. 61/935,095, filed on Feb. 3, 2014.
(60) Provisional application No. 61/874,765, filed on Sep. 6, 2013.

(51) Int. Cl. C12Q 1/68

(52) U.S. Cl. CPC C12Q 1/6886; C12Q 2600/156
USPC 424/159.1; 435/6.12; 514/44 A; 514/44 R; 424/174.1

(57) ABSTRACT

Described are biomarkers for neoplastic disease progression. Specifically, provided are methods of determining neoplastic disease progression by determining the presence of retrotransposon insertion.

### CROSS-REFERENCE TO RELATED APPLICATIONS

The present application claims priority to, and the benefit under 35 U.S.C. §119(e) of U.S. provisional patent application No. 61/874,765, filed Sep. 6, 2013 and to U.S. provisional patent application No. 61/935,095, filed Feb. 3, 2014. The entire teachings of these applications are incorporated herein by reference.

### STATEMENT OF RIGHTS TO INVENTIONS MADE UNDER FEDERALLY SPONSORED RESEARCH

This work was supported by grant number 1R01GM099875-01, awarded by the National Institutes of Health. The government has certain rights in the invention.

### FIELD OF THE INVENTION

This invention relates generally to the field of biomarkers for neoplastic disease progression and cancer treatment.

### BACKGROUND OF THE INVENTION

Prior to the invention described herein, there was a pressing need to develop new strategies for the prediction of the emergence of carcinoma from preneoplastic lesions and the emergence of metastases from primary cancer. There was also a pressing need for the development of a highly specific cancer treatment strategy, and considering treatment of cancer patients based on their retrotransposon insertion profile.

### SUMMARY OF THE INVENTION

The invention is based on the surprising discovery that somatic retrotransposon insertions serve as biomarkers for neoplastic disease progression. Specifically, described herein is the somatic retrotransposon mobilization in two gastrointestinal tumor types: colorectal cancer and pancreatic cancer. Most insertions detectable from bulk tissue deoxyribonucleic acid (DNA) are predicted to be present in most or all cells of the primary gastrointestinal cancers, and they are often present in the matched preneoplastic lesions or in matched metastases. Based on their presence exclusively in non-normal tissue, their early integration during the tumorigenic process, their presence in matched neoplastic tissue, and frequent insertional mutagenesis of cancer-related genes, many of these insertions may play a causative role in tumorigenesis. Thus, described herein is the use of retrotransposon insertions as biomarkers for neoplastic disease progression, as determinants of conventional cancer treatment strategies, and for two types of somatic retrotransposon-specific gene therapy approaches: (1) excision of etiologically significant insertions from tumor tissue by targeting their 5′ and 3′ junctions; and (2) retrotransposon insertion-specific suicide gene therapy. Viruses that have integrated into the genome are also excised or their host cell killed by suicide gene therapy according to the methods described herein.

Also described herein is the utilization of next generation sequencing to identify somatic retrotransposon mobilization in cancers and their preneoplastic lesions. Normal tissues are also mapped to identify the presence of germline insertions in these patients. As described herein, the identification of both somatic and germline retrotransposon insertions that disrupt gene regions that play a role in tumorigenesis or therapeutic outcome allows for the development of personalized cancer therapy.

Provided are methods of determining the progression of a preneoplastic lesion into a primary cancer or the progression of a primary cancer into a cancer metastasis or cancer recurrence in a subject by providing a sample from the preneoplastic lesion, from the primary cancer, or from the metastasis in the subject, detecting in the sample a biomarker comprising a somatic retrotransposon insertion, wherein the presence of the somatic retrotransposon insertion in the sample indicates that if a preneoplastic lesion is likely to progress into a primary cancer or that the primary cancer is likely to progress into a cancer metastasis, then the transposon insertion will still be present in the more “evolved” neoplastic lesion as well. For example, the progression of the preneoplastic lesion or primary cancer is monitored by providing a sample from the subject. Suitable test samples include a biological fluid selected from the group consisting of whole blood, serum, plasma, urine, pancreatic cyst fluid, and pancreatic juice.

In some cases, the differential detection of a retrotransposon insertion in a specific biological fluid is an indication of a specific biological phenomenon. For instance, the presence or change in quantity of DNA from a tumor-specific somatic retrotransposon insertion in a blood sample indicates whether the preneoplastic lesion progressed into a primary cancer or whether the primary cancer progressed into a metastasis.

Somatic retrotransposon insertions are primarily identified utilizing any convenient high throughput DNA mapping method and then are confirmed, e.g., by polymerase chain reaction (PCR).

The subject is preferably a mammal, e.g., a mammal that has been diagnosed with cancer or a predisposition thereto. The mammal is any mammal, e.g., a human, a primate, a mouse, a rat, a dog, a cat, a horse, as well as livestock or animals grown for food consumption, e.g., cattle, sheep, pigs, chickens, and goats. In a preferred embodiment, the mammal is a human.

The primary cancer is any cancer, e.g., breast cancer, cervical cancer, colon/rectum cancer, endometrial cancer, esophagus cancer, liver cancer, lung cancer, lymphoma, ovarian cancer, pancreatic cancer, penile cancer, prostate cancer, skin cancer, testicular cancer, or vaginal cancer. Preferably, the primary cancer is an epithelial cancer, e.g., a gastrointestinal cancer selected from colorectal cancer and pancreatic cancer. In some cases, the preneoplastic lesion is a colorectal polyp, an adenoma, or an inflammatory bowel disease dysplasia.

Preferably, the somatic retrotransposon insertion is absent from non-tumor, i.e., normal, tissue. For example, the somatic retrotransposon insertion is a clonal insertion in a tumor. In some aspects, the somatic retrotransposon comprises long interspersed element-1 (L1). Optionally, the somatic retrotransposon further comprises an Alu retrotransposon, an SVA retrotransposon, a processed pseudogene, an inactive retrotransposon, or a retrotransposed small ribonucleic acid (RNA) species. The somatic retrotransposon insertion comprises between 50 base pairs and 6,100 base pairs, e.g., about 100, 200, 300, 400, 500, 600, 700, 800, 900, 1,000, 1,500, 2,000, 2,500, 3,000, 3,500, 4,000, 4,500, 5,000, 5,500, or 6,000 base pairs. In some cases, an insertion may be longer than 6,100 base pairs, if, for instance, a composite retroelement is formed. Additionally, a human endogenous retrovirus (HERV) may be up to 11 kilobases (kb) long.

A method of inhibiting a tumor in a subject is carried out by providing a tumor sample from the subject, detecting in the tumor sample a biomarker comprising a somatic retrotransposon (e.g., a somatic tumor-drive retrotransposon), and excising the somatic retrotransposon from the tumor in the subject, thereby inhibiting the tumor in the subject (i.e., reversing the tumor phenotype). For example, the somatic retrotransposon is excised from the tumor in the subject by contacting the tumor in the subject with a site specific nuclease or a recombinase that specifically recognizes a 5′ or a 3′ junction of the somatic retrotransposon. Suitable site specific nucleases include a zinc-finger nuclease, a transcription activator-like effector nuclease (TALENs), and a clustered regulatory interspaced short palindromic repeat (CRISPR)/Cas-based RNA-guided DNA endonuclease. Preferably, the somatic retrotransposon is a clonal insertion.

Also provided are methods of inhibiting a tumor in a subject by providing a tumor cell from the subject, detecting in the tumor cell a biomarker comprising a somatic retrotransposon, contacting the somatic retrotransposon from the tumor in the subject with a toxic compound linked to a DNA binding domain of a site-specific nuclease or recombinase that specifically recognizes a 5′ or a 3′ junction of the somatic retrotransposon, and killing the tumor cell, thereby inhibiting the tumor in the subject. Preferably, the somatic retrotransposon insertion is absent from non-tumor tissue. By attaching a toxin to the DNA binding domain of a gene therapy construct recognizing the somatic retrotransposon, tumor cells which contain the somatic retrotransposon insertion are killed. In some cases, the somatic retrotransposon insertion is a passenger mutation.

This approach also pertains to treating tissues other than tumor tissues, i.e., normal tissue. Methods of eliminating a virus from a subject are carried out by providing a sample from a subject, detecting in the sample a virus integrated into the genome of the cell, and excising the virus from the subject, thereby eliminating virus from the subject. Preferably, the virus is a virus integrated into genomic DNA. In one aspect, the virus is a somatic human endogenous retrovirus (HERV).

This approach also pertains to treating tissues other than tumor tissues, except if elimination of that tissue would be lethal to the organism. Also provided are methods of eliminating a virus by providing a sample from the subject, detecting in the sample a virus integrated into the genome of a cell, contacting the virus from the tumor in the subject with a toxic compound linked to a DNA binding domain of a site-specific nuclease or recombinase that specifically recognizes a 5′ or a 3′ junction of the virus; and killing the cell containing the virus, thereby eliminating virus in the subject.

Methods of inhibiting a tumor in a subject are carried out by providing a tumor cell from said subject; detecting in the tumor cell a biomarker comprising a retrotransposon insertion in a gene; characterizing the retrotransposon insertion as harmful to gene function or therapeutic outcome; rejecting harmful or ineffective tumor therapy; and selecting and administering a tumor therapy, thereby inhibiting the tumor in the subject.

Suitable retrotransposons include somatic retrotransposon and germline retrotransposons. Exemplary tumor therapy includes surgery, chemotherapy, radiation therapy, nanotherapy (i.e., the utilization of nanoparticles for delivery), and gene therapy. In one aspect, therapy is administered utilizing viral delivery techniques or nanotechnology. In some cases, the tumor therapy is an agonist or antagonist of the gene, i.e., the tumor therapy inhibits or restores gene function. For example, gene therapy is utilized to introduce DNA, RNA, or complementary DNA (cDNA) that will express a functional, therapeutic gene to replace a mutated gene. Alternatively, the tumor therapy comprises an inhibitor of the gene or an inhibitor of an RNA or a protein encoded by the gene. For instance, the gene is inhibited with a small molecule inhibitor or RNA interference technology (RNAi). Alternatively, the protein is inhibited with an antibody or other compound known to inhibit the expression or function of the protein, e.g., a small molecule or a drug. In some cases, the gene plays a role in tumorigenesis.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

By the terms “effective amount” and “therapeutically effective amount” of a formulation or formulation component is meant a sufficient amount of the formulation or component, alone or in a combination, to provide the desired effect. For example, by “an effective amount” is meant an amount of a compound, alone or in a combination, required to reduce or prevent cancer in a mammal. Ultimately, the attending physician or veterinarian decides the appropriate amount and dosage regimen.

The terms “treating” and “treatment” as used herein refer to the administration of an agent or formulation to a clinically symptomatic individual afflicted with an adverse condition, disorder, or disease, so as to effect a reduction in severity and/or frequency of symptoms, eliminate the symptoms and/or their underlying cause, and/or facilitate improvement or remediation of damage.

The terms “preventing” and “prevention” refer to the administration of an agent or composition to a clinically asymptomatic individual who is susceptible or predisposed to a particular adverse condition, disorder, or disease, and thus relates to the prevention of the occurrence of symptoms and/or their underlying cause.

The term “antibody” or “immunoglobulin” is intended to encompass both polyclonal and monoclonal antibodies. The preferred antibody is a monoclonal antibody reactive with the antigen. The term “antibody” is also intended to encompass mixtures of more than one antibody reactive with the antigen (e.g., a cocktail of different types of monoclonal antibodies reactive with the antigen). The term “antibody” is further intended to encompass whole antibodies, biologically functional fragments thereof, single-chain antibodies, and genetically altered antibodies such as chimeric antibodies comprising portions from more than one species, bifunctional antibodies, antibody conjugates, humanized and human antibodies. Biologically functional antibody fragments, which can also be used, are those peptide fragments derived from an antibody that are sufficient for binding to the antigen. “Antibody” as used herein is meant to include the entire antibody as well as any antibody fragments (e.g. F(ab′)2, Fab′, Fab, Fv) capable of binding the epitope, antigen or antigenic fragment of interest.

A small molecule is a compound that is less than 2000 daltons in mass. The molecular mass of the small molecule is preferably less than 1000 daltons, more preferably less than 600 daltons, e.g., the compound is less than 500 daltons, 400 daltons, 300 daltons, 200 daltons, or 100 daltons.

Other features and advantages of the invention will be apparent from the following description of the preferred embodiments thereof, and from the claims. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All published foreign patents and patent applications cited herein are incorporated herein by reference. Genbank and NCBI submissions indicated by accession number cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

### BRIEF DESCRIPTION OF THE DRAWINGS

FIG. 1 is a photograph of an agarose gel showing a PCR validation scheme of L1-seq results. Left panel: PCR validation of a tumor-and-metastasis-specific insertion in a patient with colon polyps and tumors (ins. E8). Right panel: verification of a dysplasia-and-tumor-specific insertion (ins. C7) in an IBD patient. The higher molecular weight bands visible above the non-normal tissues of the empty site PCR products are the highly truncated L1 elements, as assessed by gel extraction and Sanger sequencing. Abbreviations: N, normal; P, polyp (adenoma); T, tumor (primary cancer, adenocarcinoma); M, metastasis; D, IBD dysplasia; (FS) filled site PCR product (insertion allele); (ES) empty site PCR product (wild type allele).

### DETAILED DESCRIPTION

Three classes of retroelements are active and a source of human disease: long interspersed elements (LINEs), the prototype of which is the RNA polymerase II transcribed L1; short interspersed elements (SINEs), consisting essentially of RNA polymerase III transcribed Alus; and SVAs (SINE-R/VNTR/Alus) that are intermediate in size relative to Alus and L1s, and are likely transcribed by RNA polymerase II. A fourth class of retroelements in the human genome, human endogenous retroviruses (HERVs) is considered immobile. Full-length L1s are not only responsible for mobilizing themselves, but also for mobilizing the nonautonomous Alu and SVA retrotransposons, inactive L1s, small RNAs, and classical mRNAs, thereby creating processed pseudogenes.

Active mobile elements are not only a significant source of intra- and interindividual variation, but can also act as insertional mutagens. There are 97 known disease-associated retrotransposon insertions into protein-coding genes, and of these 25 are caused by L1s, 60 by Alus, eight by SVAs, and four by poly(A) sequence originating from an unidentifiable source. Of these insertions, 30 occur in cancer cases, including four in colon cancer patients. In addition to acting as insertional mutagens, retrotransposons disrupt gene function and genomic integrity in many other ways, e.g., recombination-mediated gene rearrangements, genetic instability, transcriptional interference, alternative splicing, gene breaking, epigenetic effects, the generation of DNA doublestrand breaks, and the expression of small noncoding RNAs. Retrotransposon overdose is another potential scenario in malignancy and could result in increased insertional mutagenesis, toxicity, or other oncogenic effects. Indeed, the overexpression of L1 ORF1p was observed in certain tumors, and RNAi-mediated silencing of L1 s resulted in reduced proliferation and differentiation of tumorigenic cell lines. In addition, overexpression of Alu elements may exert disease through RNA toxicity (Kaneko et al. 2011).

Provided herein is high throughput mapping of preneoplastic and/or neoplastic lesions of patients for the presence of somatic retrotransposon insertions. Normal tissues are also mapped in order to identify germline insertions or somatic insertions that arose early during development and are present in large quantities in one or more normal tissues. Epithelial tumors are permissive for somatic retrotransposon mobilization. Specifically, gastrointestinal tumors allow the accumulation of these insertions, while normal tissues do not. However, normal tissues may allow a small number of insertions as ascertained from bulk tissue DNA. For example, only one normal colon-specific insertion was identified (and verified with nested PCR) in 7 colorectal cancer patient samples. As described herein, single cell sequencing identifies whether individual normal cells allow more retrotransposition events than what is detectable from bulk tissue DNA.

This invention pertains to all kinds of cancers and premalignant lesions in which somatic retrotransposon insertions are detected. Described herein is the utilization of L1-seq (Ewing and Kazazian, 2010 Genome Research, 20:1262-1270; Solyom et al., 2012 Genome Research, 22:2328-2338) to map human-specific L1 (L1Hs) insertions in cancer, but whole genome sequencing (WGS) and any kind of retroelement resequencing method, microarray-based technology, or applicable 2^{nd}/3^{rd}/further generation high throughput sequencing technique may be utilized to discover new somatic retroelement insertions in tumors. In some cases, these insertions—in a broader sense—may include other retroelements mobilized and integrated by active L1s into the tumor genome, including Alu retrotransposons, SVA retrotransposons, processed pseudogenes, inactive retrotransposons, small and large RNA species, and human endogenous retroviruses (HERVs).

These retroelement insertions are then validated by PCR to make sure they are genuine and that they are absent from normal tissues. When different sections of the same tumor and a complete sample set representing the developmental stage of tumorigenesis is available, such as pre-neoplastic lesion, primary cancer, and matched metastasis of the same patient, a spatio-temporal map of the insertions is drawn showing the timing of insertions and whether they are present in all cells of the given sample. When all or nearly all of the cells contain an insertion, it is called a “clonal event” and it likely appeared early during the tumorigenic process. The clonality of the events needs to be confirmed by an appropriate method such as a comprehensive sampling strategy to analyze multiple sections of the tumor far away from each other, digital PCR to quantify the number of wild type and insertion alleles, single cell sequencing of cells from different locations of the tumor, etc. Importantly, if several insertions are found both in the primary cancer and the metastasis, those insertions are most likely all clonal in the primary cancer, as the metastasis is presumably formed from a single cell or a few cells of the primary cancer. The clonality of the insertions will be crucial for gene therapy and desirable for the use of retrotransposon insertions as biomarkers. Importantly, as described in detail below, about half of the insertions were detected to arise in preneoplastic lesions (adenomas and IBD dysplasias were examined in colorectal cancer patients), and the majority of these insertions present in preneoplastic lesions are also present in the paired primary cancers, and insertions present in primary cancers are also detected in the paired metastases. The spatio-temporal map of the insertions is also used to identify genes/genomic intervals that are insertionally mutagenized and thus to pinpoint insertions that are potentially cancer driver events. Their etiological role (if unknown) in tumorigenesis is confirmed by functional genetic assays. Subsequently, these clonal insertions are exploited as (1) biomarkers for disease progression and (2) for cancer treatment as follows.

### Retrotransposons as Biomarkers

As described above, when an insertion is present in a premalignant lesion, it is most often found in the cancer that is formed from the premalignant lesion. Similarly, when an insertion is found in a primary cancer, it is most often present in the emerging metastasis as well. This serves the basis of retrotransposon profiling-based molecular diagnostics and the use of these insertions as biomarkers to predict neoplastic disease progression. For example, after a patient is diagnosed to have a premalignant lesion, the lesion is screened and confirmed for the presence of somatic retrotransposon insertions. The patient is subsequently monitored for the emergence of a primary cancer from the original preneoplastic lesion by sampling body fluids periodically. Methods include conventional PCR, nested PCR, quantitative PCR, or digital PCR with primers designed on the 5′ or 3′ junction. Alternatively, the retrotransposon junction can be mapped using high-throughput next generation sequencing and bioinformatics. Suitable biological fluids include whole blood, serum, and plasma. Tumor cells and circulating tumor DNA are detectable in blood, thus, if upon periodic blood sampling and PCR amplification of the 3′ or 5′ junction of one or more retrotransposon insertions are positive, or their quantity increased, it will signal that the preneoplastic lesion progressed into cancer, and that the patient urgently needs surgery or therapy. Similarly, by measuring the quantitative difference of the number of insertion alleles from body fluids, the emergence of metastases are detected. Likewise, the effectiveness of therapy is monitored for tumor shrinkage or for the recurrence of the primary tumor or metastasis upon treatment. For heightened specificity, multiple insertions and both insertion junctions are monitored by PCR or any convenient method.

### Gene Therapy

In contrast to classical mutations (e.g., point mutations and small indels), L1 insertions are large. Even though all L1 insertions detected in tumors to date are heavily truncated, the mean insertion size is about 600 bp. Importantly, any kind of retroelement insertion has the following unique advantages for specific targeting by gene therapy compared to classical mutations: they are big, providing longer specific sequence to be targeted and both their 5′ and 3′ junctions can be targeted at the same time for extra specificity. Targeting is to be achieved by zinc-finger nucleases (ZFNs), transcription activator-like effector nucleases (TALENs), clustered regulatory interspaced short palindromic repeat (CRISPR)/Cas-based RNA-guided DNA endonucleases, site-specific recombinases, and any of their derivatives or related technologies that can be designed to recognize the unique DNA sequence of a new somatic retrotransposon insertion and the adjacent unique genomic sequence junction. Since these insertions are somatic and tumor-specific, such DNA targeting is expected to be highly specific. Two types of gene therapy inventions of somatic retroelement insertions are described herein: i) excising clonal cancer driver insertions and ii) suicide gene therapy.

Excision of clonal cancer driver insertions is performed by targeting either the 5′ or the 3′ junction or both at the same time. Since these insertions are a priori demonstrated to have an etiological role in tumorigenesis and they are clonal, the removal of one or more insertions from all/most cells of the tumor reverts the malignant phenotype in the patient. Potential side effects need to be minimized, and targeting efficiency needs to be high.

For suicide gene therapy, only the DNA binding domains of the above-described genetic engineering constructs are used. Instead of coupling them to the nuclease or recombinase domain, a toxic compound is linked to the DNA binding domains. Alternatively, a signaling molecule initiates cell death. In some cases, the toxin has two domains, wherein one domain attaches to the insertion's 5′ junction via the first DNA binding domain, and the other domain of the toxic compound attaches to the insertion's 3′ junction via the second DNA binding domain. The toxic compound becomes complete or activated only when the linked DNA binding domains specifically bind both to the 5′ and 3′ junction and the two half-toxins form a complete toxin. For this kind of gene therapy, it is not essential that the insertions have a cancer driver function. Rather, it is enough if they are present in the tumor (even as passenger mutations), but absent from normal cells. Any (pre)neoplastic cell containing somatic retroelement insertions may be subjected to this therapy and should be killed, leaving normal cells intact. Potential side effects need to be minimized, and targeting efficiency needs to be high.

As described herein, viruses that integrate into the genome are excised or their host cells are killed in the same way as described for retroelement insertions above.

Prior to the invention described herein, the presence of retrotransposon insertions was not shown in preneoplastic lesions (i.e. colorectal polyps and inflammatory bowel disease dysplasias). Prior to the invention, it was also not known that these insertions are present both in preneoplastic lesions and their matched primary cancers, as well as both in primary cancers and their matched metastases, or in different sections of the same tumor. These results suggest early, clonal insertions in the sense that the insertions are predicted to be present in most, if not all cells of the tumors.

As described herein, based on the strong likelihood that the insertions are marking all cells of the tumor, somatic retrotransposon insertions are used as biomarkers for neoplastic disease progression. Precisely, if certain somatic retrotransposon insertions are present in a preneoplastic lesion, the majority of the insertions will be present in the emerging carcinoma. The same is true for insertions present in metastasis formed from the primary cancer. Thus, described herein is the utilization of somatic retrotransposon insertions as biomarkers for progression of a preneoplastic lesion into primary cancer and the progression of primary cancer into metastases, as well as to monitor disease recurrence. Also provided herein are gene therapy methods, wherein etiologically significant (cancer driver) clonal insertions are excised from the tumors using site-specific nucleases or recombinases specifically recognizing the insertions' 5′ and/or 3′ junctions, which is predicted to reverse the malignant phenotype. In some cases, clonal somatic retrotransposon insertions are targeted by a toxic compound linked to a DNA binding domain of a site-specific nuclease or recombinase specifically recognizing the insertions' 5′ and 3′ junctions. The toxic compound is designed to kill the cells which contain the given somatic retrotransposon insertion(s). This suicide gene therapy strategy is effective even if the insertions are passenger mutations, i.e., etiologically not significant for tumorigenesis. This approach is feasible because the insertions are only present in the pre-neoplastic or neoplastic cells, but are absent from normal tissue.

Prior to the invention described herein, neither somatic tumor-specific retrotransposon-based diagnostics, nor the treatment of patients with cancer or preneoplastic lesions containing somatic retrotransposon insertions have been proposed with retrotransposon insertion-specific nucleases/recombinases and linked toxic compounds. Specifically, Solyom S, et al. 2012 Genome Res, 22:2328-2338 described retrotransposition insertions in colorectal tumors; however, this reference did not describe insertions in preneoplastic lesions or any of the treatment methods described herein.

Prior to the invention described herein, personalized treatment options were developed for cancer patients based on classical mutations in genes with an established role in tumorigenesis or response to therapy. However, these mutation profiles were incomplete as they did not consider somatic and germline retroelement insertions. The impact of retrotransposon insertions on the selection of conventional therapeutic interventions was not previously considered. Thus, prior to the invention described herein, important gene targets for therapy were not identified and/or therapy not well-suited for the patient was selected.

As described herein, next generation sequencing is utilized to identify somatic retrotransposon mobilization in cancers and their preneoplastic lesions. Normal tissues are also mapped to identify the presence of germline insertions in these patients. Numerous insertions mutagenize cancer-related genes, and play a role in tumorigenesis. These insertions are not identified by conventional genetic methods or by next generation sequencing techniques that are not tailor-made to detect these elements. Thus, described herein is the identification of both somatic and germline retrotransposon insertions that disrupt gene regions which play a role in tumorigenesis or therapeutic outcome. Otherwise, the underlying genetic region may not be identified as a target for personalized cancer therapy.

### EXAMPLE 1

### Somatic L1 Insertions in Patients with Cancer

As shown in Table 1, PCR-verified somatic L1 insertions in 4 patients with colon polyps and cancers (two of the 4 patients had metastases) (top panel), from 5 IBD patients with colon dysplasias and carcinomas (middle panel), and from 7 patients with pancreatic carcinomas and metastases (bottom panel). Only top quality L1-seq reads have been validated so far, and the real number of somatic insertions is expected to be order of magnitudes higher. Note that in contrast to the paired polyp-cancer samples, at least some IBD cancers were immediately adjacent to, and likely originated from, their matched dysplasias. The tumor in patient 3BV has been reclassified as adenoma with high grade dysplasia. Blue: very early insertion events in premalignant lesions; red: potentially clonal and likewise early insertion events. Abbreviations: N, normal; P, polyp; C, primary cancer; C1, cancer section 1; C2, cancer section 2; M, metastasis; D, IBD dysplasia.

TABLE 1

| Patient ID | N-only | P-only | C-only | M-only | P + C | P + M | C + M | P + C + M | |
|---|---|---|---|---|---|---|---|---|---|
| 1BV | 0 | 0 | 13 | no M | 0 | no M | no M | no M | |
| 2BV | 1 | 10 | 1 | 2 | 0 | 0 | 4 | 0 | |
| 3BV | 0 | 0 | 17 | no M | 0 | no M | no M | no M | |
| 4BV | 0 | 1 | 0 | 0 | 0 | 0 | 7 | 0 | |
| total: | 1 | 11 | 31 | 2 | 0 | 0 | 11 | 0 | total: 56 |

| Patient ID | N-only | D-only | C-only | D + C | |
|---|---|---|---|---|---|
| H26 | 0 | 0 | 4 | 0 | |
| H28 | 0 | 1 | 0 | 0 | |
| H69 | 0 | 2 | 4 | 0 | |
| H145 | 0 | 1 | 0 | 7 | |
| H147 | 0 | 0 | 2 | 0 | |
| total: | 0 | 4 | 10 | 7 | total: 21 |

| Patient ID | N-only | C1-only | C2-only | C1 + C2 | M-only | C + M | |
|---|---|---|---|---|---|---|---|
| A33 | 0 | 0 | 0 | 0 | 2 | 1 | |
| A43 | 0 | 0 | 0 | 0 | 2 | 0 | |
| A55 | 0 | 0 | 0 | 0 | 2 | 3 | |
| A57 | 0 | PanIn: 0 | 0 | 0 | 1 | 2 | |
| A82 | 0 | 0 | 0 | 0 | 0 | 0 | |
| A83 | 0 | 0 | 0 | 1 | 0 | 3 | |
| A146 | 0 | 0 | 0 | 0 | no M | no M | |
| total: | 0 | 0 | 0 | 1 | 7 | 9 | total: 17 |

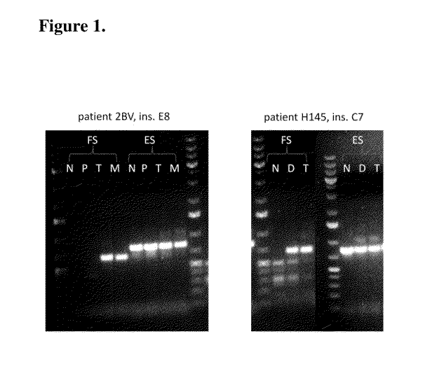

As shown in FIG. 1, PCR was utilized as a validation scheme of L1-seq results. The left panel shows PCR validation of a tumor-and-metastasis-specific insertion in a patient with colon polyps and tumors (ins. E8), while the right panel shows verification of a dysplasia-and-tumor-specific insertion (ins. C7) in an IBD patient. The higher molecular weight bands visible above the non-normal tissues of the empty site PCR products are the highly truncated L1 elements, as assessed by gel extraction and Sanger sequencing. Abbreviations: N, normal; P, polyp; T, tumor; M, metastasis; D, IBD dysplasia; (FS) filled site PCR product (insertion allele); (ES) empty site PCR product (wild type allele). Similar results have been obtained in pancreatic carcinoma and metastasis cases.

### EXAMPLE 2

### Treatment of Cancer Patients Based on Their Retrotransposon Insertion Profile

Next generation sequencing is utilized to identify somatic retrotransposon mobilization in cancers and their preneoplastic lesions. Normal tissues are also mapped to identify the presence of germline insertions in these patients. Specifically, described herein is the identification of both somatic and germline retrotransposon insertions that disrupt gene regions that play a role in tumorigenesis or therapeutic outcome.

The identified retroelement insertions are validated by PCR to ensure they are genuine, absent from normal tissues (for somatic tumor-specific insertions), or present in normal tissues (for germline insertions). In some cases, retrotransposon insertions are more harmful than classical insertions. However, since personalized therapies have not previously accounted for these retrotransposon insertions, if the functional effects of specific retrotransposon insertions are not clear, functional assays (e.g., with cell lines) are utilized to determine the effect of the retrotransposon insertion on gene function. If the insertion is identified as being harmful to gene function, depending on the gene, the correct therapeutic intervention is selected.

Any classical chemotherapeutic agent or radiation therapy is utilized in the methods described herein. For example, if the insertion mutagenizes a cancer-driver gene, and a drug exists that blocks its RNA or protein product or re-stabilizes the involved pathway, said drug should be considered for treatment. As another example, if the insertion mutagenizes a gene that renders resistance to the therapy initially chosen for patient treatment, that therapy should not be pursued, and a better therapeutic option should be favored. As another example, if a germline insertion mutagenizes a gene that renders the patient sensitive to radiation or chemotherapy, the therapy should not be pursued, as it would likely kill the patient.

For example, patients with Kirsten rat sarcoma viral oncogene homolog (K-RAS) mutant cancer are inherently resistant to antibody-based epidermal growth factor receptor (EGFR) inhibitors. As such, the identification in a cancer patient of an L1 retrotransposon insertion that mutagenizes the K-RAS gene and effects its function indicates that the patient should not be administered antibody-based EGFR inhibitors, as they would be ineffective. Specifically, cetuximab (an EGFR inhibitor mAb) may be considered as chemotherapy in a colorectal cancer patient. If said patient has an activating retroelement insertion into his/her K-RAS gene, cetuximab treatment will be most likely ineffective. Very similarly, loss of PTEN activity, due to an inactivating L1 insertion will result in the lack of efficacy of cetuximab. In either case, cetuximab should not be used and a better treatment option needs to be considered.

As another example, described herein is the identification of a somatic primary colorectal cancer-and-metastasis-specific intronic L1 insertion into the CYLD gene that encodes a deubiquitinating enzyme and is mutated in cylindromatosis. Also described herein is a somatic primary pancreatic cancer-and-metastasis-specific intronic L1 insertion into the APAF1 gene (apoptotic peptidase activating factor 1). The APAF1 gene initiates apoptosis, is a component of the apoptosome, and is dysregulated in pancreatic ductal adenocarcinomas). The effect of each of these insertions on gene function is analyzed to assist in the identification of the correct therapeutic intervention. Both deubiquitinating enzymes and APAF1 are targets for pharmacological intervention, as demonstrated by the number of compounds and assays used to develop inhibitors or activators of these proteins. See, e.g., http://www.ncbi.nlm.nih.gov/pcassay/?term=cyld (incorporated herein by reference) and http://www.ncbi.nlm.nih.gov/pcassay/?term=apafl (incorporated herein by reference).

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of determining the progression of a preneoplastic lesion into a primary cancer or the progression of a primary cancer into a cancer metastasis or the effectiveness of the cancer therapy or cancer recurrence in a subject comprising:
providing a sample from said preneoplastic lesion, from said primary cancer, or from said metastasis in said subject;
detecting in said preneoplastic lesion, primary cancer, or metastasis sample a biomarker comprising a somatic retrotransposon insertion; and
monitoring the progression of said preneoplastic lesion, primary cancer, or metastasis by providing a sample from said subject, wherein the presence of said somatic retrotransposon insertion in said sample indicates whether said preneoplastic lesion progressed into a primary cancer, whether said primary cancer progressed into a metastasis, whether cancer responded to therapy, or whether regression occurred.

2. The method of claim 1, wherein said sample is selected from the group consisting of whole blood, serum, plasma, urine, pancreatic cyst fluid, and pancreatic juice.

3. The method of claim 1, wherein said subject is a human subject.

4. The method of claim 1, wherein said primary cancer is breast cancer, cervical cancer, colon/rectum cancer, endometrial cancer, esophagus cancer, liver cancer, lung cancer, lymphoma, ovarian cancer, pancreatic cancer, penile cancer, prostate cancer, skin cancer, testicular cancer, or vaginal cancer.

5. The method of claim 1, wherein said primary cancer is an epithelial cancer.

6. The method of claim 1, wherein said cancer is a gastrointestinal cancer selected from colorectal cancer and pancreatic cancer.

7. The method of claim 1, wherein said preneoplastic lesion is a colorectal polyp, an adenoma, or an inflammatory bowel disease dysplasia.

8. The method of claim 1, wherein said somatic retrotransposon insertion is absent from non-tumor tissue.

9. The method of claim 1, wherein said somatic retrotransposon comprises long interspersed element-1 (L1).

10. The method of claim 9, wherein said somatic retrotransposon further comprises an Alu retrotransposon, an SVA retrotransposon, a processed pseudogene, an inactive retrotransposon, or a small RNA species.

11. The method of claim 1, wherein said somatic retrotransposon insertion is a clonal insertion.

12. The method of claim 1, wherein said somatic retrotransposon insertion comprises between 100 base pairs and 6,100 base pairs.

13. A method of inhibiting a tumor in a subject comprising:
providing a tumor sample from said subject;
detecting in said tumor sample a biomarker comprising a somatic tumor-driver retrotransposon; and
excising said somatic retrotransposon from said tumor in said subject, thereby inhibiting said tumor in said subject.

14. The method of claim 13, wherein said somatic retrotransposon is excised from said tumor in said subject by contacting said tumor in said subject with a site specific nuclease or a recombinase that specifically recognizes a 5′ or a 3′ junction of said somatic retrotransposon.

15. The method of claim 14, wherein said site specific nuclease comprises a zinc-finger nuclease, a transcription activator-like effector nuclease (TALENs), or a clustered regulatory interspaced short palindromic repeat (CRISPR)/Cas-based RNA-guided DNA endonuclease.

16. The method of claim 13, wherein said somatic retrotransposon insertion is a clonal insertion.

17. The method of claim 13, wherein said somatic retrotransposon comprises long interspersed element-1 (L1).

18. A method of inhibiting a tumor in a subject comprising:
providing a tumor cell from said subject;
detecting in said tumor cell a biomarker comprising a somatic retrotransposon;
contacting said somatic retrotransposon from said tumor in said subject with a toxic compound linked to a DNA binding domain of a site-specific nuclease or recombinase that specifically recognizes a 5′ or a 3′ junction of said somatic retrotransposon; and
killing said tumor cell,
thereby inhibiting said tumor in said subject.

19. The method of claim 18, wherein said somatic retrotransposon insertion is absent from non-tumor tissue.

20. A method of eliminating virus in a subject comprising:
providing a sample from said subject;
detecting in said sample a virus integrated into the genome of said cell; and
excising said virus from said subject,
thereby eliminating virus in said subject.

21. The method of claim 20, wherein said virus is a virus integrated into genomic DNA.

22. The method of claim 20, wherein said virus is a human endogenous retrovirus (HERV).

23. A method of eliminating virus in a subject comprising:
providing a sample from said subject;
detecting in said sample a virus integrated into the genome of a cell;
contacting said virus in said subject with a toxic compound linked to a DNA binding domain of a site-specific nuclease or recombinase that specifically recognizes a 5′ or a 3′ junction of said virus; and
killing said cell containing said virus,
thereby eliminating virus in said subject.

24. A method of inhibiting a tumor in a subject comprising:
providing a tumor cell from said subject;
detecting in said tumor cell a biomarker comprising a retrotransposon insertion in a gene;
characterizing said retrotransposon insertion as harmful to gene function or therapeutic outcome;
rejecting harmful or ineffective tumor therapy;
selecting and administering a tumor therapy; and
thereby inhibiting said tumor in said subject.

25. The method of claim 24, wherein said tumor therapy is surgery, chemotherapy, radiation therapy, nanotherapy, or gene therapy.

26. The method of claim 24, wherein said tumor therapy is an agonist or antagonist of said gene.

27. The method of claim 24, wherein said tumor therapy inhibits or restores gene function.

28. The method of claim 24, wherein said tumor therapy comprises an inhibitor of said gene, an inhibitor of an RNA encoded by said gene, or an inhibitor of a protein encoded by said gene.

29. The method of claim 24, wherein said retrotransposon is a somatic retrotransposon or a germline retrotransposon.

30. The method of claim 28, wherein said gene is inhibited with a small molecule inhibitor, RNAi, gene therapy, or a drug.

31. The method of claim 28, wherein said protein is inhibited with an antibody or a small molecule.

32. The method of claim 24, wherein said gene plays a role in tumorigenesis.
